(19) United States
(12) Patent Application Publication
(10) Pub. No.: US 2005/0231103 A1
(43) Pub. Date: Oct. 20, 2005

(54) Flat panel display device and method for manufacturing the same

(75) Inventors: Hoon Kim, Suwon-si (KR); Jae-Hoon Chung, Suwon-si (KR); Nam-Deog Kim, Yongin-si (KR)

Correspondence Address: MCGUIREWOODS, LLP, 1750 TYSONS BLVD, SUITE 1800, MCLEAN, VA, 22102, US

(73) Assignee: Samsung Electronics, Inc.

(21) Appl. No.: 11/081,747

(22) Filed: Mar. 17, 2005

(30) Foreign Application Priority Data
Apr. 17, 2004 (KR) 2004-26407

(51) Int. Cl. H05B 33/00

(52) U.S. Cl. 313/504

(57) ABSTRACT

A flat panel display includes a substrate, an organic light emitting element, a low melting point metal layer and an inorganic composite layer. The organic light emitting element disposed on the substrate includes a first electrode, a second electrode opposing to the first electrode and an organic light emitting layer disposed between the first and second electrodes to generate a light when current flows through the organic light emitting layer. The low melting point metal layer is disposed on the organic light emitting element to protect the organic light emitting element. The inorganic composite layer having a plurality of inorganic substances mixed with one another is disposed on the low melting point metal layer to protect the low melting point metal layer and the organic light emitting element. Therefore, the organic light emitting element is improved, and a manufacturing process of the flat panel display is simplified.

### CROSS-REFERENCE OF RELATED APPLICATIONS

The present application claims priority from Korean Patent Application No. 2004-26407, filed on Apr. 17, 2004, the disclosure of which is hereby incorporated herein by reference in its entirety.

### BACKGROUND OF THE INVENTION

1. Field of the Invention

The present invention relates to a flat panel display device and a method for manufacturing the flat panel display device. Particularly, the present invention relates to a flat panel display device that effectively protects an organic light emitting element and can reduce the manufacturing cost. The present invention relates also to a method of manufacturing the flat panel display device.

2. Description of the Related Art

A flat panel display is classified into a liquid crystal display (LCD), a plasma panel display (PDP), an organic light emitting display (OLED), etc.

The LCD device includes an additional light source, which increase thickness of the LCD. Also, the viewing angle of LCD is narrow. The PDP consumes power a lot.

On the other hand, the OLED device shows better characteristics, such as high luminance, wide viewing angle, thin profile, low power consumption and so on. In addition, the OLED device can go through a simpler manufacturing process, reducing the manufacturing costs. Furthermore, the OLED made on a flexible substrate can provide a flexible display device, which is in big demand.

The organic light emitting element of the OLED includes a pixel electrode, a counter electrode and an organic light emitting layer. The pixel electrode and the counter electrode supply holes and electrons, respectively. When an electron and a hole are injected into the organic light emitting layer from the two electrodes, respectively. The OLED display device generates an exciton by coupling the electron to the hole, and generates light when the exciton changes from an excitation state to a ground state.

Exposure of the organic light emitting layer of the OLED to water or oxygen deteriorates the electrochemical characteristics of the organic light emitting layer. Therefore, the OLED requires a closed space or a protection layer to insulate the organic light emitting element from the water or the oxygen.

For this purpose, the OLED formed a metallic can or a glass plate on the organic light emitting element. However, the metallic can or the glass plate complicated the manufacturing process and increased the manufacturing cost of the OLED. Also, it increased the OLED thickness.

An organic or inorganic layer is coated on the organic light emitting element layer to form a protection layer. Plasma, heat or ultraviolet used in forming the protection layer, however, may deteriorate the organic light emitting element.

Protection layers formed through different processes tend to delay the manufacturing process and increase the manufacturing cost.

The thermal distortion may also deteriorate elements of other flat panel display, such as LCD, or PDP.

### BRIEF SUMMARY OF THE INVENTION

The present invention provides a flat panel display apparatus that effectively protects its light emitting element and can reduce the manufacturing cost.

The present invention also provides a simplified method of manufacturing the flat panel display.

The flat panel display of an exemplary embodiment of the present invention includes a substrate, an organic light emitting element, a metal layer and an inorganic composite layer. The organic light emitting element is disposed on the substrate. The organic light emitting element includes a first electrode, a second electrode and an organic light emitting layer. The second electrode faces the first electrode with organic light emitting layer inserted therebetween. A low melting point metal layer is formed on the organic light emitting element as protection layer. An inorganic composite layer may be formed on the low melting point metal layer to protect the low melting point metal layer and the organic light emitting element. The inorganic composite layer includes an inorganic composite comprising a plurality of inorganic substances that are mixed with one another.

The low melting point metal layer includes lithium (Li), zinc (Zn), gallium (Ga), rubidium (Rb), cesium (Cs), thallium (TI), bismuth (Bi), tin (Sn), indium (In), sodium (Na), potassium (K) or an alloy having a mixture thereof. A melting point of the low melting point metal layer may be no higher than about 300° C. In some instances, the melting point may also be no higher than about 150° C.

The present invention also provides a method for manufacturing a flat panel display. A first electrode, an organic light emitting layer and a second electrode are formed on a substrate to form an organic light emitting element. The organic light emitting element generates a light in response to a current flow. The second electrode faces the first electrode with the organic light emitting layer. A low melting point metal is deposited on the organic light emitting element. An inorganic composite that includes a plurality of inorganic substances that are mixed with one another is formed on the low melting point metal.

Another aspect of the present invention deposits and forms the low melting point metal and the inorganic composite material on the organic light emitting element in a chamber in-situ.

The low melting point metal layer may include the alloy so that the low melting point metal layer may be formed at a temperature of no higher than about 150° C. In addition, the alloy may prevent a crystallization of the inorganic composite layer deposited thereon. Furthermore, the inorganic composite layer includes the inorganic composite material having the inorganic substances that can decrease the inorganic composite layer's permeability. Also, the low melting point metal layer and the inorganic composite layer are formed in the same chamber in-situ to simplify the manufacturing process and to reduce the manufacturing time of the flat panel display.

The flat panel display may include an organic light emitting display (OLED). The OLED may include an active type OLED and a passive type OLED.

### BRIEF DESCRIPTION OF THE DRAWINGS

The above and other features of the present invention will become more apparent by describing in detail exemplary embodiments thereof with reference to the accompanying drawings.

FIG. 1 is a plan view showing a flat panel display in accordance with an exemplary embodiment of the present invention.

FIG. 2 is a cross-sectional view taken along the line I-I′ of FIG. 1.

FIGS. 3, 4, 5 and 6 are cross-sectional views showing a method for manufacturing a flat panel display in accordance with an exemplary embodiment of the present invention.

FIG. 7 is a cross-sectional view showing a thermal evaporation for depositing a low melting point metal and an inorganic composite material in accordance with an exemplary embodiment of the present invention.

FIG. 8 is a cross-sectional view showing a flat panel display in accordance with another exemplary embodiment of the present invention.

FIG. 9 is a cross-sectional view showing a flat panel display in accordance with another exemplary embodiment of the present invention.

FIGS. 10, 11 and 12 are cross-sectional views showing a method for manufacturing a flat panel display in accordance with another exemplary embodiment of the present invention.

FIG. 13 is a cross-sectional view showing a flat panel display in accordance with another exemplary embodiment of the present invention.

FIGS. 14, 15, 16 and 17 are cross-sectional views showing a method for manufacturing a flat panel display in accordance with another exemplary embodiment of the present invention.

### DESCRIPTION OF PREFERRED EMBODIMENTS

It should be understood that the exemplary embodiments of the present invention described below may be varied modified in many different ways without departing from the inventive principles disclosed herein, and the scope of the present invention is therefore not limited to these particular following embodiments. Rather, these embodiments are provided so that this disclosure will be thorough and complete, and will fully convey the concept of the invention to those skilled in the art by way of example and not of limitation.

The following will describe the present invention in detail with reference to the accompanying drawings.

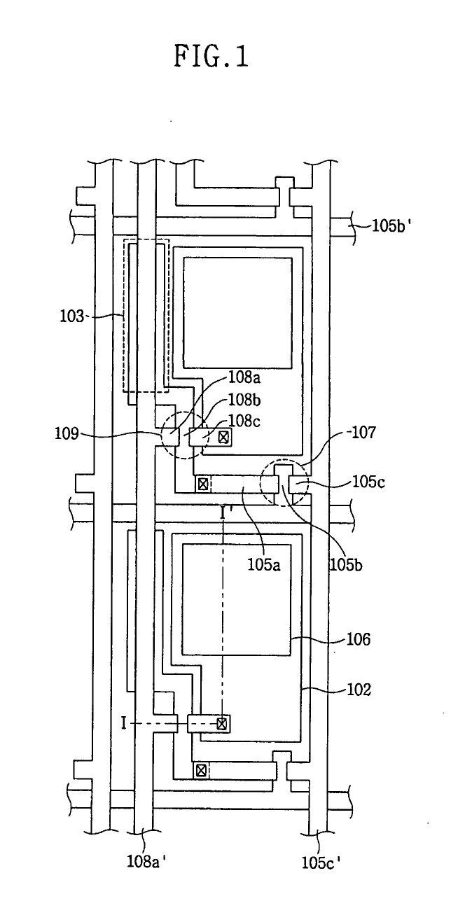

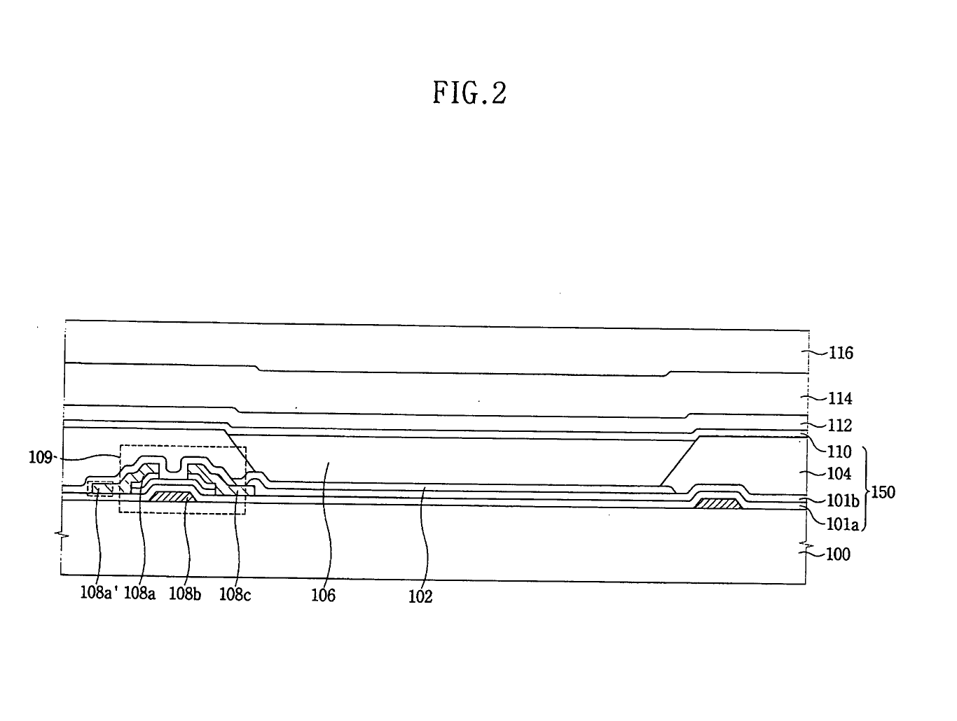

FIG. 1 is a plan view showing a flat panel display in accordance with an exemplary embodiment of the present invention. FIG. 2 is a cross-sectional view taken along the line I-I′ of FIG. 1.

Referring to FIGS. 1 and 2, the flat panel display includes a substrate 100, an organic light emitting element 150, a storage capacitor 103, a low melting point metal layer 112, an inorganic composite layer 114 and an organic protection layer 116.

The substrate 100 includes glass, triacetylcellulose (TAC), polycarbonate (PC), polyethersulfone (PES), polyethyleneterephthalate (PET), polyethylenenaphthalate (PEN), polyvinylalcohol (PVA), polymethylmethacrylate (PMMA), cyclo-olefin polymer (COP) or a mixture thereof.

A pixel includes an organic light emitting element 150, a switching transistor 107, a driving transistor 109, a gate insulating layer 101a, an insulating layer 101b. The organic light emitting element 150 includes a pixel electrode 102, a bank 104, an organic light emitting layer 106, and a counter electrode 110.

The switching transistor 107 includes a first source electrode 105c, a first gate electrode 105b, a first drain electrode 105a and a first semiconductor layer pattern (not shown). The first source electrode 105c is electrically connected to a data line 105c′ to receive a data signal outputted from a driving circuit (not shown). The first gate electrode 105b is disposed on the substrate 100 to be coupled to a gate line 105b′ to receive a gate voltage outputted from the driving circuit. The first drain electrode 105a is spaced apart from the first source electrode 105c. The first semiconductor layer pattern (not shown) is disposed between the first drain electrode 105a and the first source electrode 105c.

The driving transistor 109 includes a second drain electrode 108a, a second gate electrode 108b and a second source electrode 108c. The second drain electrode 108a is electrically connected to a bias line 108a′ to receive a bias voltage. The second gate electrode 108b is disposed on the substrate 100. The second gate electrode 108b is electrically connected to the first drain electrode 105a of the switching transistor 107 through an auxiliary contact hole. The second source electrode 108c is spaced apart from the second drain electrode 108a. The second semiconductor pattern not shown is disposed between the second source electrode 108c and the second drain electrode 108a.

When the data voltage and the gate voltage are applied to the data line 105c′ and the gate line 105b′, respectively, the data voltage is applied to the second gate electrode 108b through the first source electrode 105c, the first semiconductor pattern and the first drain electrode 105a. When the data voltage is applied to the second gate electrode 108b, a channel is formed in the second semiconductor layer pattern so that the bias voltage is applied to the second source electrode 108c.

The gate insulating layer 101a electrically insulates the first gate electrode 105b, the gate line 105b′ and the second gate electrode 108b from the first source electrode 105a, the data line 105c′, the first drain electrode 105c, the second drain electrode 108a, the bias line 108a′ and the second source electrode 108c. The gate insulating layer 101a includes an insulating material, for example, such as silicon nitride, silicon oxide, etc.

The insulating layer 101b is disposed on the substrate 100 having the switching transistor 107, the driving transistor 109, the gate line 105b′, the data line 105c′ and the bias line 108a′. The insulating layer 101b includes a contact hole, through which the second source electrode 108c is electrically connected to the pixel electrode 102. The insulating layer 101b includes an organic insulating material or an inorganic insulating material, for example, such as the silicon nitride, the silicon oxide.

A portion of the second gate electrode 108b overlaps with a portion of the bias line 108a′ to form the storage capacitor 103. The storage capacitor 103 maintains a voltage difference between the pixel electrode 102 and the counter electrode 110 during a frame.

The pixel electrode 102 is disposed on the substrate 100 in a region defined by the bias line 108a′, the gate line 105b′ and the data line 105c′. The pixel electrode 102 includes a transparent conductive material, for example, such as indium tin oxide (ITO), indium zinc oxide (IZO), zinc oxide (ZO), etc.

The bank 104 is disposed on the insulating layer 101b having the pixel electrode 102 to form a recessed portion on a central portion of the pixel electrode 102.

The organic light emitting layer 106 is formed in the recessed portion formed by the bank 104. The organic light emitting layer 106 may includes tris(8-hydroxy-quinolate)aluminum (Alq3), polyparavinyl, polyfluorene. The organic light emitting layer 106 includes a red organic light emitting portion, a green organic light emitting portion and a blue organic light emitting portion. The red organic light emitting portion may include a dopant, for example, such as dichloromethane (DCM), DCJT, DCJTB, etc. The green organic light emitting layer may include a dopant, for example, such as coumarin 6, quinacridone (Qd), etc.

The counter electrode 110 is formed on the organic light emitting layer 106 and the bank 104. A common voltage is applied to the counter electrode 110. The counter electrode 110 includes a metal oxide or a metal, for example, such as calcium (Ca), barium (Ba), aluminum (Al), etc. Alternatively, the counter electrode 110 may include a conductive material having low permeability to protect the organic light emitting layer 106.

In this exemplary embodiment, the pixel electrode 102 includes a transparent conductive material. Alternatively, the counter electrode 110 may include the transparent conductive material.

The bias voltage applied to the second drain electrode 108c is applied to the pixel electrode 102 through the contact hole. Therefore, a current flows between the pixel electrode 102 and the counter electrode 110 through the organic light emitting layer 108. Holes supplied from the pixel electrode 102 are combined with electrons supplied from the counter electrode 110 to form an exciton in the organic light emitting layer 106. When the exciton decays into ground state losing its energy, which in turn generates lights.

The low melting point metal layer 112 is disposed on the organic light emitting element 150 to protect it from a heat generated in subsequent processes. The low melting point metal layer 112 may also protect the organic light emitting element 150 from ambient impurities. The impurities may include, for example, water and oxygen that may deteriorate the organic light emitting element 150. A melting point of the low melting point metal layer 112 may be no higher than about 300° C. at an atmospheric pressure. Preferably, the melting point of the low melting point metal layer 112 may be no higher than about 150° C. at the atmospheric pressure. The organic light emitting element 150 may change its characteristics at a temperature higher than about 150° C. In addition, a temperature higher than about 300° C. may significantly deteriorate the organic light emitting element 150.

The low melting point metal layer 112 includes a low melting point metal, for example, such as lithium (Li), zinc (Zn), gallium (Ga), rubidium (Rb), cesium (Cs), thallium (TI), bismuth (Bi), tin (Sn), indium (In), sodium (Na), potassium (K), etc. The low melting point metal layer 112 may include an alloy having a plurality of the low melting point metals.

A melting point of the lithium is about 280.69° C. (553.69 K) at the atmospheric pressure. A melting point of the zinc is about 420.73° C. (692.73 K) at the atmospheric pressure. A melting point of the gallium is about 29.93° C. (302.93 K) at the atmospheric pressure. A melting point of the rubidium is about 39.2° C. (312.2 K) at the atmospheric pressure. A melting point of the cesium is about 28.6° C. (301.6 K) at the atmospheric pressure. A melting point of the thallium is about 303.6° C. (576.6 K) at the atmospheric pressure. A melting point of the bismuth is about 271.5° C. (544.5 K) at the atmospheric pressure. A melting point of the tin is about 232.1° C. (505.1 K) at the atmospheric pressure. A melting point of the indium is about 156.32° C. (429.32 K) at the atmospheric pressure. A melting point of the sodium is about 97.96° C. (370.96 K) at the atmospheric pressure. A melting point of the potassium is about 63.8° C. (336.8 K) at the atmospheric pressure. Forming the low melting point metal layer 112 in a vacuum atmosphere decreases the melting point of the low melting point metal.

The melting point of the low melting point metal layer 112 may be no higher than about 150° C. Alternatively, the counter electrode 110 may be omitted so that the common voltage may be applied to the low melting point metal layer 112. The low melting point metal 112 may be thicker than about 10 nm.

The inorganic composite layer 114 is formed on the low melting point metal layer 112 to protect the low melting point metal layer 112 and the organic light emitting element 150 from the impurities from the outside of the flat panel display. The impurities deteriorate electrical/optical characteristics of the organic light emitting element 150. The inorganic composite layer 114 protects the low melting point metal layer 112 and the organic light emitting element 150 from the heat generated in the subsequent processes.

The inorganic composite layer 114 includes a plurality of inorganic substances mixed with one another. The inorganic substances include at least one material from the group of silicon oxide, silicon carbide, lithium oxide, magnesium oxide, calcium oxide, barium oxide, silica gel, aluminum oxide, titanium oxide, silicon oxynitride, silicon nitride, aluminum nitride, magnesium fluoride, and activated carbon. The inorganic composite layer 114 may be about 50 nm to about 500 μm thick.

The inorganic composite layer 114 includes inorganic molecules having different sizes. The inorganic molecules with different sizes are packed to reduce a permeability of the inorganic composite layer 114 less than an inorganic layer having one inorganic substance.

The organic protection layer 116 is disposed on the inorganic composite layer 114 to protect the inorganic composite layer 114, the low melting point metal layer 112 and the organic light emitting element 150 from an impact that is provided from the exterior to the flat panel display. The organic protection layer 116 includes a polymer resin, parylene, etc. The polymer resin has low permeability. The polymer resin includes epoxy, silicone, fluoric resin, acrylic resin, urethane resin, phenolic resin, polyethylene, polypropylene, polystyrene, polymethyl methacrylate, polyurea, polyimide or a mixture thereof.

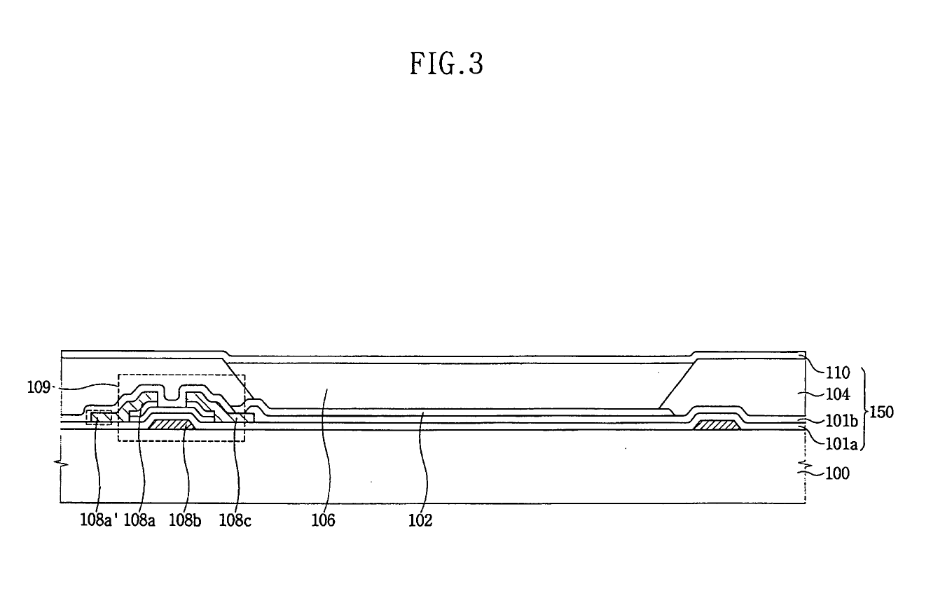

FIGS. 3, 4, 5 and 6 are cross-sectional views showing a method for manufacturing a flat panel display device in accordance with an exemplary embodiment of the present invention.

Referring to FIG. 3, a metal layer is deposited on the substrate 100 and patterned to form the first gate electrode 105b, the gate line 105b′ and the second gate electrode 108b.

The insulating material is deposited on the substrate 100 having the first gate electrode 105b, the gate line 105b′ and the second gate electrode 108b. The insulating material is etched to form the gate insulating layer 101a having the auxiliary contact hole, through which the first drain electrode 105a is electrically connected to the second gate electrode 108b.

An amorphous silicon pattern and an N+ amorphous silicon pattern are formed on the gate insulating layer 101a corresponding to the first gate electrode 105b and the second gate electrode 108b to form the first semiconductor layer pattern and the second semiconductor layer pattern.

A metal is deposited on the gate insulating layer 101a having the first and second semiconductor layer patterns thereon. The deposited metal is partially etched to form the first source electrode 105c, the data line 105c′, the first drain electrode 105a, the second drain electrode 108a, the bias line 108a′, the second source electrode 108c and the storage capacitor 103. Therefore, the switching transistor 107 and the driving transistor 109 are formed on the substrate 100. The switching transistor 107 includes the first source electrode 105c, the first gate electrode 105b, the first drain electrode 105a and the first semiconductor layer pattern. The driving transistor 109 includes the second drain electrode 108c, the second gate electrode 108b, the second source electrode 108a and the second semiconductor layer pattern.

Another insulating layer is deposited on the substrate 100 having the switching transistor 107, the driving transistor 109, the gate line 105b′, the data line 105c′ and the bias line 108a′. The insulating layer is etched to form the insulating layer 101b having the contact hole, through which the second source electrode 108c is partially exposed. The insulating layer 101b may include an inorganic layer or an organic layer.

A metal layer is deposited on the insulating layer 101b. The metal layer is etched to form the pixel electrode 102. The pixel electrode 102 is electrically coupled to the second source electrode 108c through the contact hole.

An organic material is coated on the insulating layer 101b having the pixel electrode 102 thereon. The organic material is partially removed through a photolithography process to form the bank 104.

An organic light emitting material is laid in the recessed portion between the bank 104 through an ink jet process to form an organic light emitting layer 106.

A conductive layer is formed on the organic light emitting layer 106 and the bank 104 to form a counter electrode 110.

This concludes formation of the organic light emitting element 150 on the substrate 100. The organic light emitting element 150 includes the gate insulating layer 101a, the insulating layer 101b, the pixel electrode 102, the bank 104, the organic light emitting layer 106, the switching transistor 107, the driving transistor 109 and the counter electrode 110.

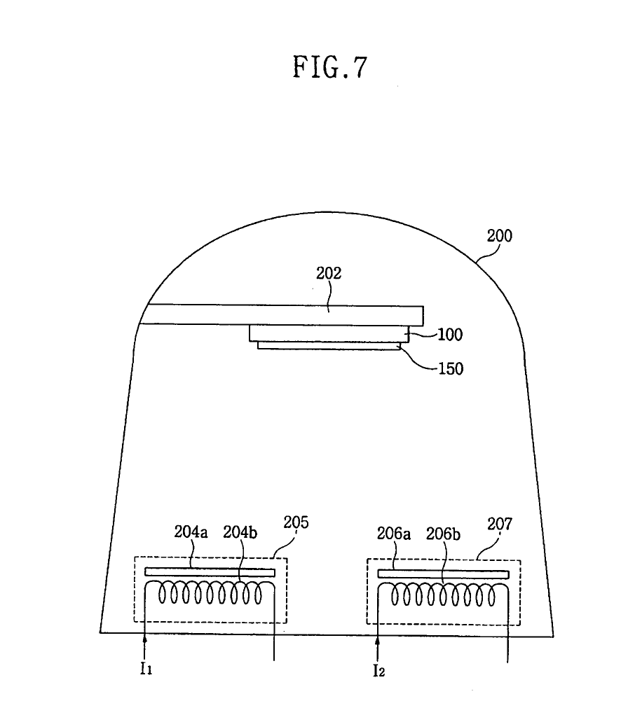

FIG. 7 is a cross-sectional view showing a thermal evaporation device for depositing a low melting point metal and an inorganic composite material in accordance with an exemplary embodiment of the present invention.

Referring to FIG. 7, the thermal evaporation device includes a chamber 200, a substrate fixing unit 202, a low melting point metal supplying unit 205 and an inorganic composite material supplying unit 207.

The substrate 100 having the organic light emitting element 150 is disposed on the substrate fixing unit 202.

The first supplying unit 205 corresponds to the substrate fixing unit 202 in the chamber 200. In the present embodiment, the first supplying unit 205 includes a first heater 204b and the low melting point metal source 204a disposed on the first heater 204b.

The second supplying unit 207 corresponds to the substrate fixing unit 202 in the chamber 200. In the present embodiment, the first supplying unit 207 is spaced apart from the first supplying unit 205. The second supplying unit 207 includes a second heater 206b and the inorganic composite material source 206a.

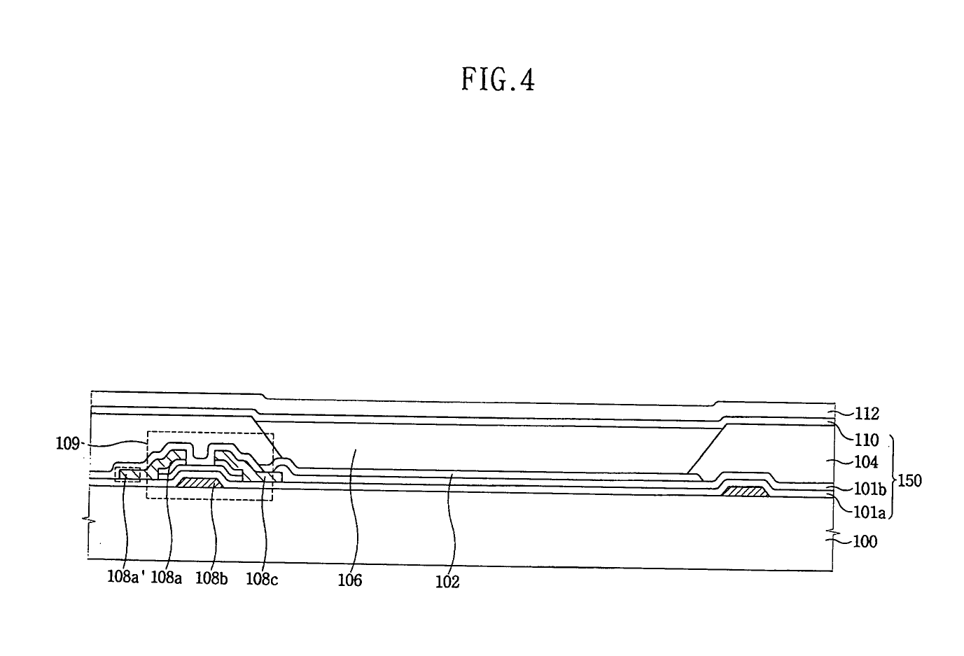

Referring to FIGS. 4 and 7, the low melting point metal heater 204b heats the low melting point metal source 204a at the temperature of no higher than 300° C. using a first current I₁, which ejects metal molecules of the low melting point metal source 204a onto the substrate 100. A portion of the ejected metal molecules is deposited on the organic light emitting element 150 to form the low melting point metal layer 112. Alternatively the low melting point metal heater 204b heats the low melting point metal source 204a at the temperature of no higher than 150° C.

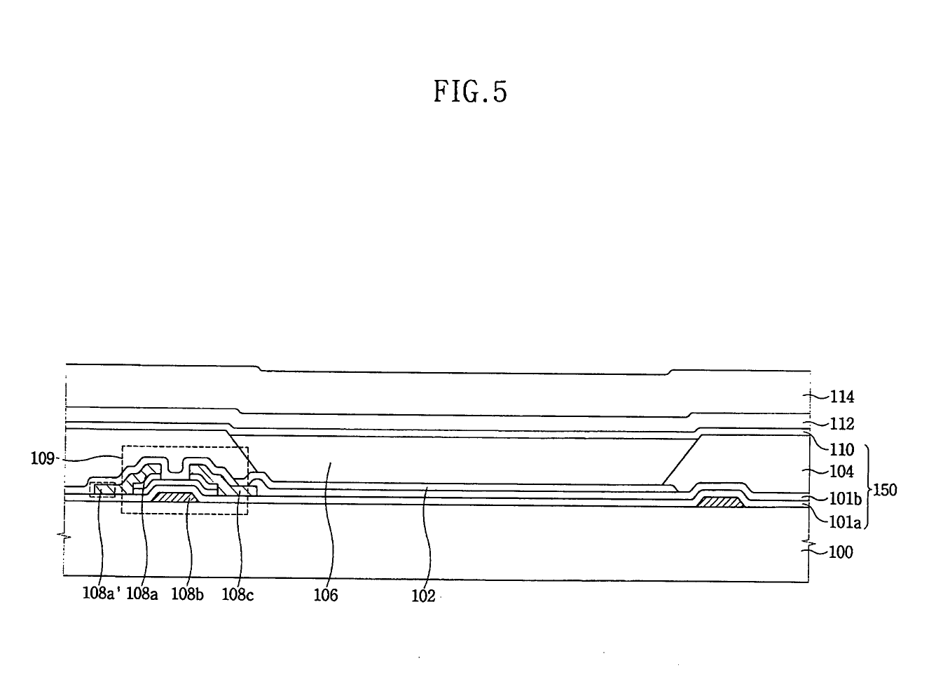

Referring to FIGS. 5 and 7, the inorganic composite material heater 206b heats the inorganic composite material source 206a using a second current I₂, which ejects inorganic molecules of the inorganic composite material source 206a onto the substrate 100. As a heating temperature of the second heater 206b increases, the permeability of the inorganic composite layer 114 decreases. The second heater 206b may heat the inorganic composite material source 206a at a temperature of no less than about 200° C. Although the second heater 206b heats the inorganic composite material source 206a at the temperature of more than about 200° C., the low melting point metal layer 112 protects the organic light emitting element 150 from the heat formed during the deposition of the inorganic composite layer 114. Alternatively, the second heater 206b may also heat the inorganic composite material source 206a at a temperature of no less than about 300° C. A portion of the ejected inorganic molecules is deposited on the low melting point metal layer 112 to form the inorganic composite layer 114.

The low melting point metal layer 112 and/or the inorganic composite layer 114 may be formed using a physical vapor deposition (PVD).

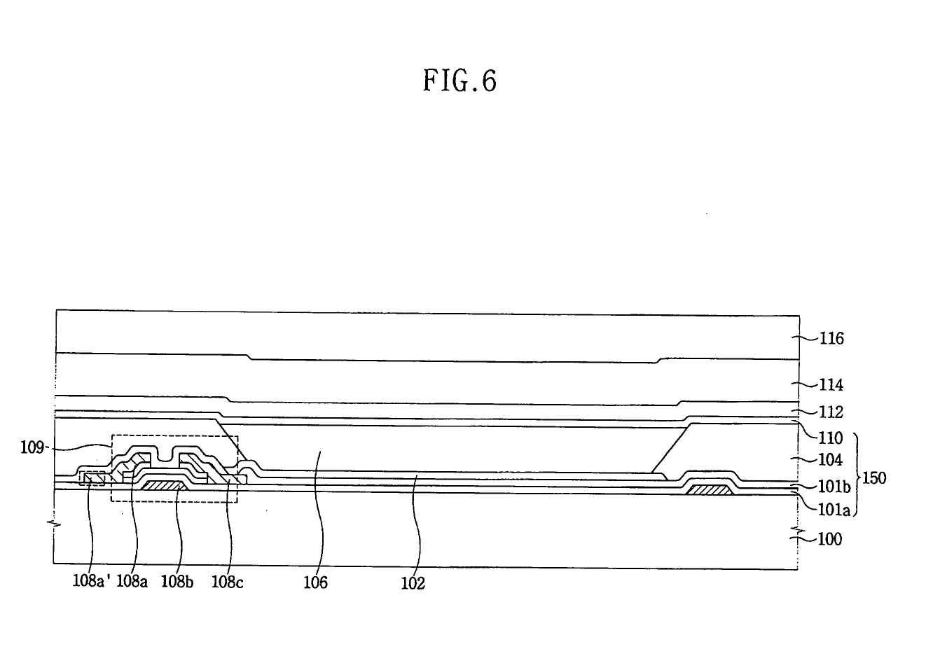

Referring to FIG. 6, the polymer resin having a low permeability is coated on the inorganic composite layer 114 to form the organic protection layer 116. The polymer resin may be coated using a screen printing process, a slit coating process, a capillary coating process, etc. Although the polymer resin is coated on the inorganic composite layer 114 at a temperature of no less than about 200° C., the inorganic composite layer 114 and the low melting point metal layer 112 protects the organic light emitting element 150 from the heat formed during the formation of the organic protection layer 116. When the polymer resin is coated at the temperature of higher than about 200° C., the permeability of the organic protection layer 116 decreases.

According to this exemplary embodiment, the low melting point metal layer 112 is formed on the counter electrode 110 to protect the organic light emitting element 150 from the heat. In addition, the inorganic composite layer 114 includes the inorganic composite material having the inorganic substances that are mixed with one another to protect the organic light emitting element 150 from the impurities.

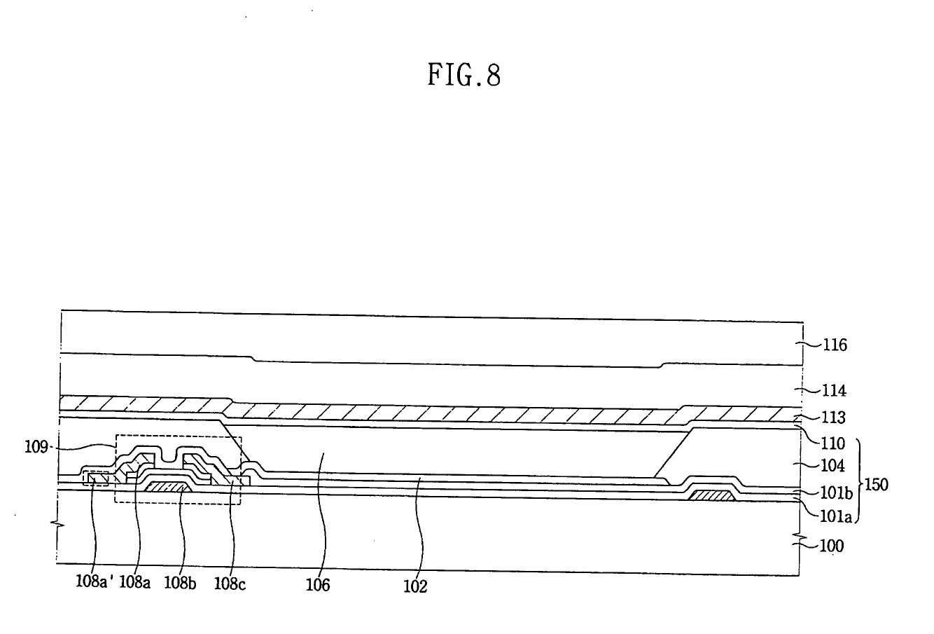

FIG. 8 is a cross-sectional view showing a flat panel display in accordance with another exemplary embodiment of the present invention. The flat panel display of FIG. 8 is same as in FIGS. 1 and 2 except a low melting point metal layer. Thus, the same reference numerals will be used to refer to the same or like parts as those described in FIGS. 1 and 2 and any repetitive explanation will be omitted.

Referring to FIGS. 1 and 8, the flat panel display includes a substrate 100, an organic light emitting element 150, a storage capacitor 103, a low melting point metal layer 113, an inorganic composite layer 114 and an organic protection layer 116.

The organic light emitting element 150 includes a gate insulating layer 101a, an insulating layer 101b, a pixel electrode 102, a bank 104, an organic light emitting layer 106, a switching transistor 107, a driving transistor 109 and a counter electrode 110.

The switching transistor 107 includes a first source electrode 105c electrically connected to a data line 105c′, a first gate electrode 105b electrically connected to a gate line 105b′, a first drain electrode 105a and a first semiconductor layer pattern (not shown).

The driving transistor 108 includes a second drain electrode 108a electrically connected to a bias line 108a′, a second gate electrode 108b electrically connected to the first drain electrode 105a of the switching transistor 107 through an auxiliary contact hole and a second source electrode 108c.

The low melting point metal layer 113 is disposed on the organic light emitting element 150 to protect the organic light emitting element 150. A melting point of the layer 113 may be no higher than about 150° C. at an atmospheric pressure.

The low melting point metal layer 113 includes an alloy having a mixture of lithium (Li), zinc (Zn), gallium (Ga), rubidium (Rb), cesium (Cs), thallium (TI), bismuth (Bi), tin (Sn), indium (In), sodium (Na) and potassium (K). A melting point of the alloy is lower than that of a pure low melting point metal.

According to this exemplary embodiment, the low melting point metal layer 113 of the above alloys can be formed at a temperature lower than the melting point of the pure low melting point metal. This prevents the organic light emitting element 150 further potential thermal stress.

In addition, metal molecules of the alloy have different sizes so that the metal molecules are packed to decrease a permeability of the low melting point metal layer 113. Furthermore, a surface of the low melting point metal layer 113 having the alloy is more irregular than that of the low melting point metal layer having the pure low melting point metal to prevent a crystallization of the inorganic composite layer 114. This decreases a permeability of the inorganic composite layer 114.

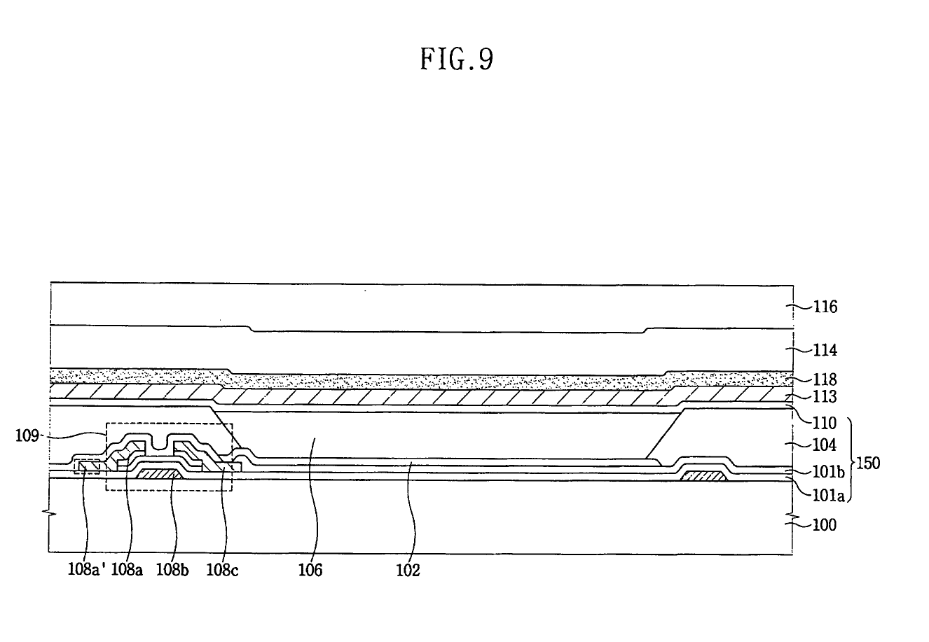

FIG. 9 is a cross-sectional view showing a flat panel display in accordance with another exemplary embodiment of the present invention. The flat panel display of FIG. 9 is is same as in FIGS. 1 and 2 except a low melting point metal layer and an absorption layer. Thus, the same reference numerals will be used to refer to the same or like parts as those described in FIGS. 1 and 2 and any repetitive explanation will be omitted.

Referring to FIGS. 1 and 9, the flat panel display includes a substrate 100, an organic light emitting element 150, a storage capacitor 103, a low melting point metal layer 113, an absorption layer 118, an inorganic composite layer 114 and an organic protection layer 116.

The organic light emitting element 150 includes a gate insulating layer 101a, an insulating layer 101b, a pixel electrode 102, a bank 104, an organic light emitting layer 106, a switching transistor 107, a driving transistor 109 and a counter electrode 110.

The switching transistor 107 includes a first source electrode 105c electrically connected to a data line 105c′, a first gate electrode 105b electrically connected to a gate line 105b′, a first drain electrode 105a and a first semiconductor layer pattern (not shown).

The driving transistor 109 includes a second drain electrode 108a electrically connected to a bias line 108a′, a second gate electrode 108b electrically connected to the first drain electrode 105a of the switching transistor 107 through an auxiliary contact hole and a second source electrode 108c.

The low melting point metal layer 113 is disposed on the organic light emitting element 150. The low melting point metal layer 113 includes an alloy having a low melting point metal, for example, such as lithium (Li), zinc (Zn), gallium (Ga), rubidium (Rb), cesium (Cs), thallium (TI), bismuth (Bi), tin (Sn), indium (In), sodium (Na), potassium (K), etc.

The absorption layer 118 is disposed on the low melting point metal layer 113 to protect the organic light emitting element 150 and the low melting point metal layer 113 from a moisture coming from ambient environment. The absorption layer 118 includes a hydroscopic material, for example, such as inorganic silica, silicon carbide, calcium oxide, barium oxide, magnesium oxide, activated carbon or a mixture thereof.

The inorganic composite layer 114 is disposed on the absorption layer 118 to protect the absorption layer 118, the low melting point metal layer 113 and the organic light emitting element 150 from an impurity from outside of flat panel display. The inorganic composite layer 114 may protect the absorption layer 118, the low melting point metal layer 113 and the organic light emitting element 150 from an outside impact.

The organic protection layer 116 is disposed on the inorganic composite layer 114 to protect the inorganic composite layer 114, the absorption layer 118, the low melting point metal layer 113 and the organic light emitting element 150 also from the outside impact. The organic protection layer 116 may also protect the inorganic composite layer 114, the absorption layer 118, the low melting point metal layer 113 and the organic light emitting element 150 from the outside impurity.

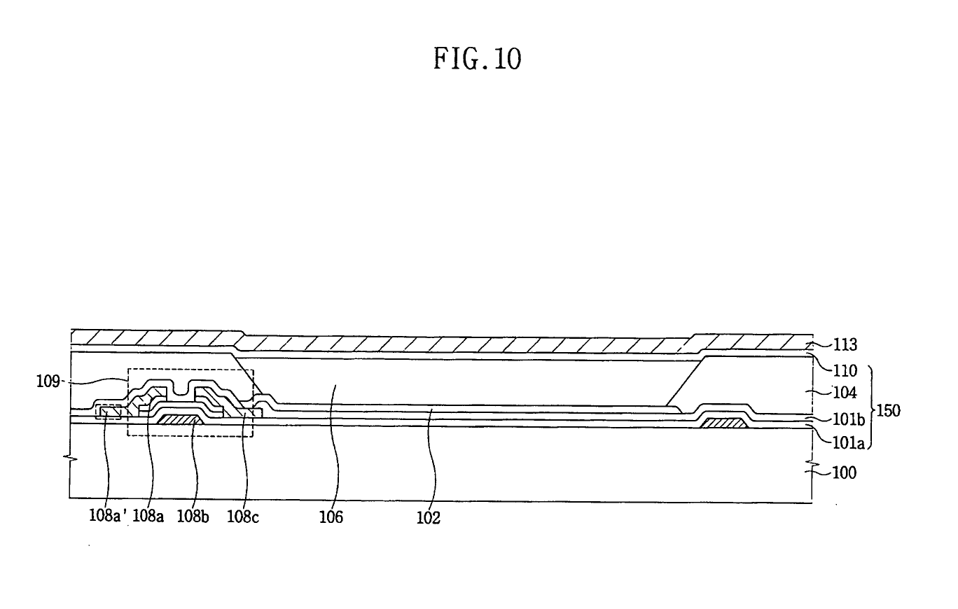

FIGS. 10, 11 and 12 are cross-sectional views showing a method for manufacturing a flat display panel in accordance with another exemplary embodiment of the present invention.

Referring to FIG. 10, the organic light emitting element 150 is formed on the substrate 100.

The low melting point metal is deposited on the organic light emitting element 150 to form the low melting point metal layer 113.

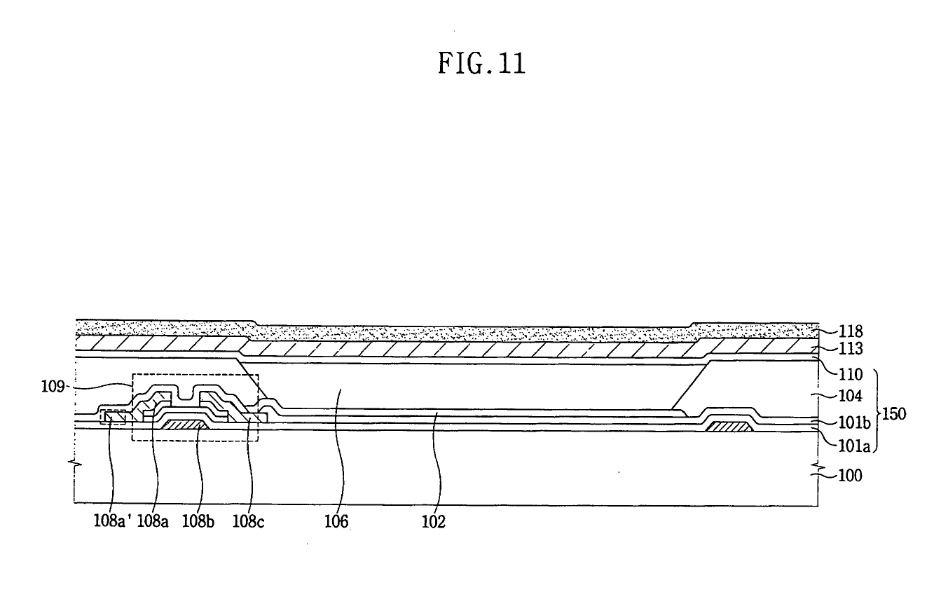

Referring to FIG. 11, the hydroscopic material is deposited on the low melting point metal layer 113 to form the absorption layer 118.

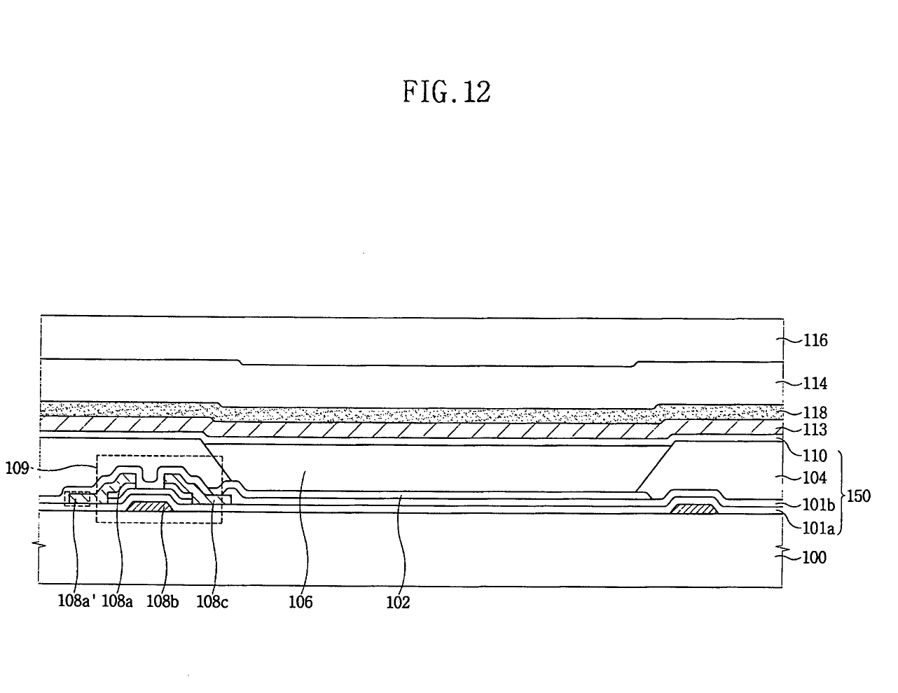

Referring to FIG. 12, the inorganic composite material is deposited on the is absorption layer 118 to form the inorganic composite layer 114.

The low melting point metal layer 113, the absorption layer 118 and the inorganic composite layer 114 may be formed through a physical vapor deposition (PVD) process, a thermal evaporation process, etc. Alternatively, the low melting point metal layer 113, the absorption layer 118 and the inorganic composite layer 114 may be deposited in-situ.

A polymer resin is coated on the inorganic composite layer 114 to form the organic protection layer 116.

According to this exemplary embodiment, the absorption layer 118 is disposed between the low melting point metal layer 113 and the inorganic composite layer 114 to protect the organic light emitting element 150 from a moisture from the outside of the flat panel display.

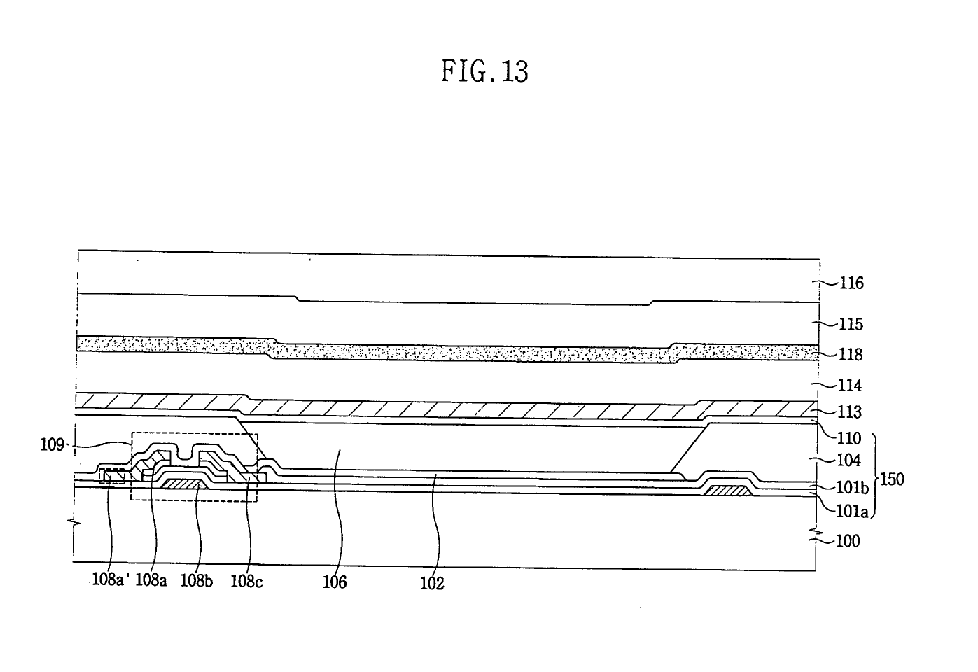

FIG. 13 is a cross-sectional view showing a flat panel display in accordance with another exemplary embodiment of the present invention. The flat panel display of FIG. 13 is same as in FIGS. 1 and 2 except a low melting point metal layer, an absorption layer and an auxiliary inorganic layer. Thus, the same reference numerals will be used to refer to the same or like parts as those described in FIGS. 1 and 2 and the same explanation will not be repeated.

Referring to FIGS. 1 and 13, the flat panel display includes a substrate 100, an organic light emitting element 150, a storage capacitor 103, a low melting point metal layer 113, an inorganic composite layer 114, an absorption layer 118, an auxiliary inorganic layer 115 and an organic protection layer 116.

In this embodiment, the low melting point metal layer 113 is disposed on the organic light emitting element 150. The low melting point metal layer 113 includes an alloy is having a plurality of low melting point metals, for example, such as lithium (Li), zinc (Zn), gallium (Ga), rubidium (Rb), cesium (Cs), thallium (TI), bismuth (Bi), tin (Sn), indium (In), sodium (Na), potassium (K), etc.

The inorganic composite layer 114 is disposed on the low melting point metal layer 113 to protect the low melting point metal layer 113 and the organic light emitting element 150 from an impurity that is provided from an exterior to the flat panel display.

The absorption layer 118 is disposed on the inorganic composite layer 114 to protect the inorganic composite layer 114, the low melting point metal layer 118 and the organic light emitting element 150 from outside moisture.

The auxiliary inorganic layer 115 is disposed on the absorption layer 118 to protect the absorption layer 118, the inorganic composite layer 114, the low melting point metal layer 113 and the organic light emitting element 150 from the outside impurity.

The auxiliary inorganic layer 115 includes an inorganic substance, for example, such as silicon oxide, silicon carbide, lithium oxide, magnesium oxide, calcium oxide, barium oxide, silica gel, aluminum oxide, titanium oxide, silicon oxynitride, silicon nitride, aluminum nitride, magnesium fluoride, activated carbon, etc. Alternatively, the auxiliary inorganic layer 115 may include an inorganic composite material having a plurality of the inorganic substances that are mixed with one another.

The organic protection layer 116 protects the auxiliary inorganic layer 115, the absorption layer 118, the inorganic composite layer 114, the low melting point metal layer 113 and the organic light emitting element 150 from an exterior impact.

In another exemplary embodiment, the flat panel display may also include a plurality of inorganic layers and/or a plurality of organic layers that are disposed on the organic is protection layer 116.

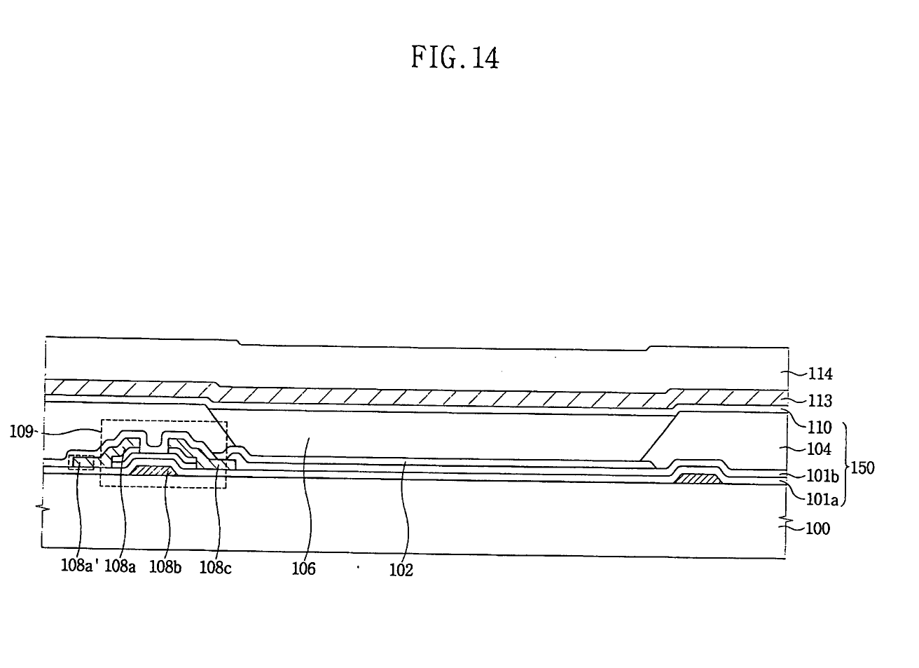

FIGS. 14, 15, 16 and 17 are cross-sectional views showing a method for manufacturing a flat panel display in accordance another exemplary embodiment of the present invention.

Referring to FIG. 14, the organic light emitting element 150 is formed on the substrate 100.

The low melting point metal is deposited on the organic light emitting element 150 to form the low melting point metal layer 113.

The inorganic composite material is deposited on the low melting point metal layer 113 to form the inorganic composite layer 114.

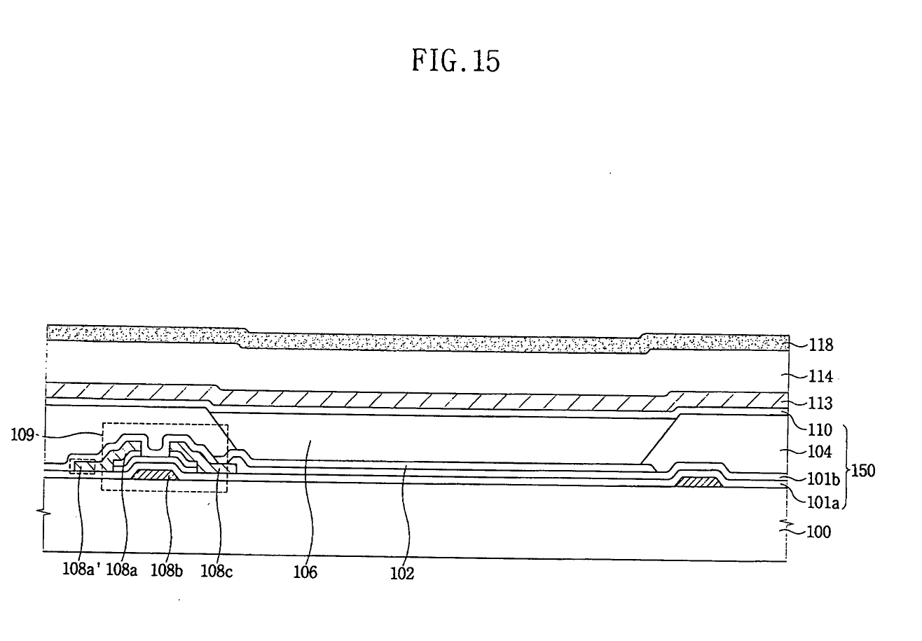

Referring to FIG. 15, the hydroscopic material is deposited on the inorganic composite layer 114 to form the absorption layer 118.

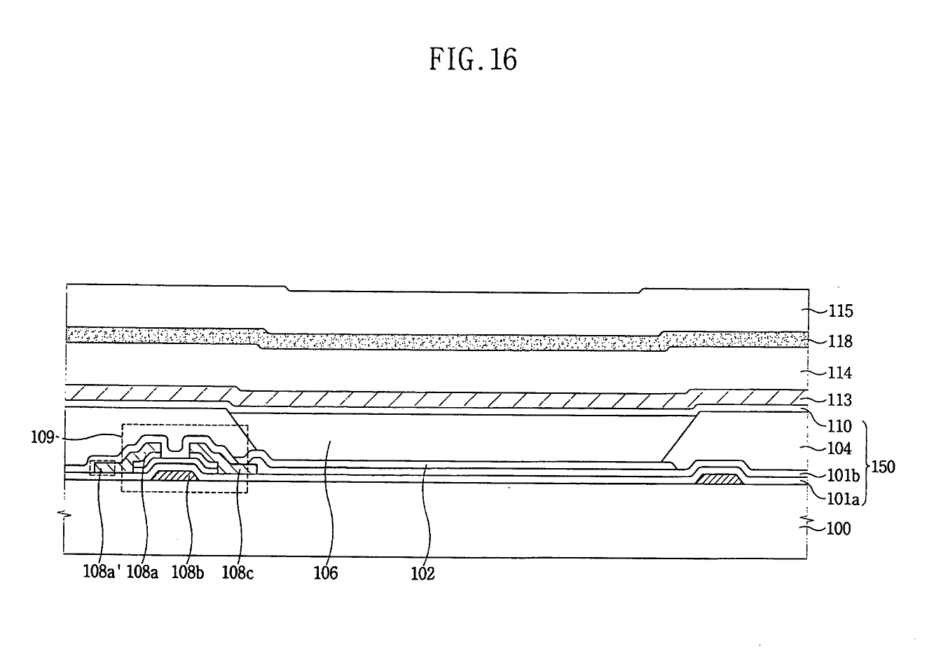

Referring to FIG. 16, the inorganic substance or the inorganic composite material is deposited on the absorption layer 118 to form the auxiliary inorganic layer 115.

The low melting point metal layer 113, the inorganic composite layer 114, the absorption layer 118 and the auxiliary inorganic layer 115 may be formed through a physical vapor deposition (PVD), a thermal evaporation process, etc. The low melting point metal layer 113, the inorganic composite layer 114, the absorption layer 118 and the auxiliary inorganic layer 115 may be deposited in-situ.

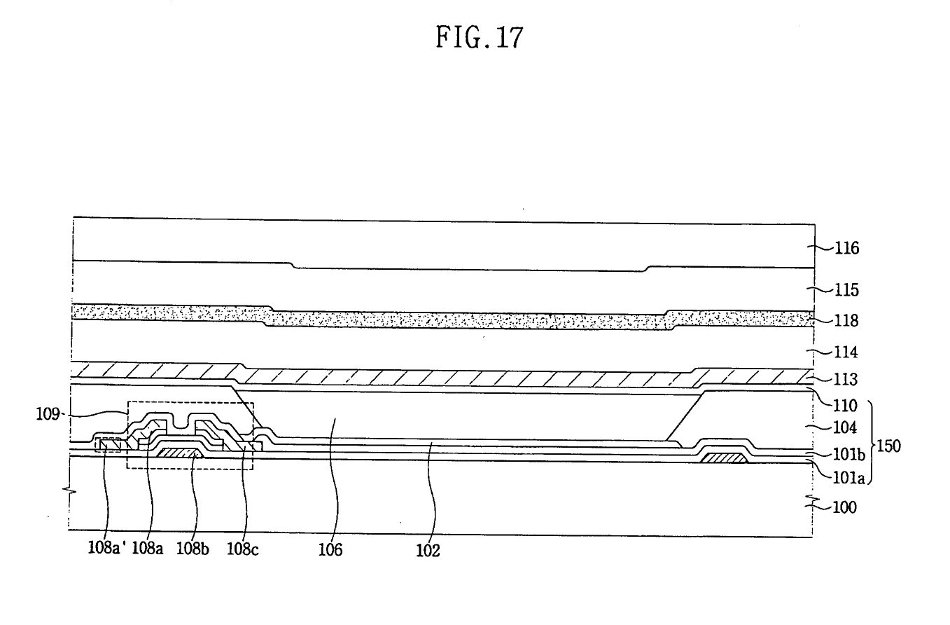

Referring to FIG. 17, a polymer resin is coated on the auxiliary inorganic layer 115 to form the organic protection layer 116.

According to this exemplary embodiment, the flat panel display includes the absorption layer 118 disposed on the inorganic composite layer 114 and the auxiliary inorganic layer 115 disposed on the absorption layer 118 to protect the organic light emitting element 150 from the outside impurity.

Although not intending to be bound by theory, one possible reason as to why an inorganic composite layer's permeability is less than that of an inorganic layer having one inorganic substance will be described. Molecular sizes of the inorganic layer having one inorganic substance are substantially equal to one another. In contrast, molecular sizes of the inorganic composite layer are different from one another. When the molecules having different sizes are mixed, small molecules are packed between large molecules to decrease the permeability of the inorganic composite layer.

In addition, molecular sizes of a low melting point metal layer having one low melting point metal are substantially equal to one another. However, molecular sizes of a low melting point metal layer having an alloy of low melting point metals are different from one another. When the molecules having different sizes are mixed, small molecules are packed between large molecules so that the low melting point metal layer having the alloy has more compact structure than the low melting point metal layer having one low melting point metal.

Furthermore, a melting point of the alloy is lower than that of a pure metal so that a melting point of the low melting point metal layer having the alloy is lower than that of the low melting point metal layer having one low melting point metal. Therefore, although the alloy includes zinc (Zn) or thallium (TI) that has a melting point of higher than about 300° C., the melting point of the low melting point metal layer having the alloy may be no higher than 150° C.

When the low melting point metal layer includes the alloy, the alloy may prevent a crystallization of the inorganic composite layer deposited on the low melting point metal layer to decrease the permeability of the inorganic composite layer. A permeability of an amorphous inorganic material, in general, is less than that of a polycrystalline inorganic material. The low melting point metal layer having the alloy includes more irregular surface than the low melting point metal layer having the one low melting point metal so that an arrangement of the molecules deposited on the low melting point metal layer having the alloy is disturbed, thereby preventing the crystallization of the inorganic composite layer deposited on the low melting point metal layer.

According to this present invention, a low melting point metal layer protects an organic light emitting element. In addition, the low melting point metal layer includes an alloy so that the low melting point metal layer may be formed at a temperature of no higher than about 150° C., and a crystallization of an inorganic composite layer deposited on the low melting point metal layer may be prevented. Furthermore, the inorganic composite layer includes an inorganic composite material having inorganic substances that are mixed with one another so that a permeability of the inorganic composite layer decreases. Although a composition of the inorganic composite material is changed, a structural mismatch formed between the inorganic composite layer and the substrate decreases. Also, a mismatch of a stress formed by a difference between thermal expansion coefficients of the inorganic composite layer and the substrate may also decrease.

Furthermore, the low melting point metal layer and the inorganic composite layer may be formed in a chamber in-situ, which simplifies a manufacturing process of the flat panel display and decreases time for manufacturing the flat panel display. This, in turn, increases a throughput of the flat panel display.

This invention has been described with reference to the exemplary embodiments. It is evident, however, that many alternative modifications and variations will be apparent to those having skill in the art in light of the foregoing description. Accordingly, the present invention embraces all such alternative modifications and variations as fall within the spirit and scope of the appended claims.

1. An organic electroluminescent device, comprising:
a substrate;
a first electrode formed on the substrate;
an organic light emitting layer formed on the first electrode;
a second electrode formed on the organic light emitting layer; and
a metal layer formed on the second electrode layer.

2. The organic electroluminescent device of claim 1, further comprising:
an inorganic layer formed on the metal layer.

3. The organic electroluminescent device of claim 2,
wherein the metal layer melts at lower than 300° C.

4. The organic electroluminescent device of claim 3,
wherein the metal layer melts at lower than 150° C.

5. The organic electroluminescent device of claim 3,
wherein the metal layer is more than 19 nm thick.

6. The organic electroluminescent device of claim 2,
wherein the inorganic layer is a composite layer.

7. The organic electroluminescent device of claim 6,
wherein the composite layer is formed of at least two kinds of inorganic substances.

8. The organic electroluminescent device of claim 2, further comprising:
an organic layer and an inorganic layer formed on the metal layer.

9. The organic electroluminescent device of claim 7,
wherein the organic layer and the inorganic layer are formed alternatively.

10. The organic electroluminescent device of claim 7, further comprising:
an absorption layer formed on the metal layer.

11. The organic electroluminescent device of claim 1,
wherein the metal layer comprises at least one of lithium (Li), zinc (Zn), gallium (Ga), ribidium (Rb), cesium (Cs), thallium (TI), bismuth (Bi), tin (Sn), indium (In), sodium (Na), potassium (K) or their alloys.

12. The organic electroluminescent device of claim 9,
wherein the absorption layer comprises at least one from the group of inorganic silica, silicon carbide, calcium oxide, barium oxide, magnesium oxide, and activated carbon.

13. The organic electroluminescent device of claim 2, further comprising:
an organic layer formed on the inorganic composite layer.

14. The organic electroluminescent device of claim 1,
wherein the metal layer is a composite of at least two among In, Sn, Ti and Zn.

15. The organic electroluminescent device of claim 13, further comprising:
an absorption layer comprising at least one from the group of inorganic silica, silicon carbide, calcium oxide, barium oxide, magnesium oxide, and activated carbon.

16. The organic electroluminescent device of claim 13, further comprising:
an organic layer that is about 50 um to about 500 um thick.

17. A method for manufacturing an organic electroluminescent device, comprising:
forming a first electrode on a substrate;
forming an organic light emitting layer on the first electrode;
forming a second electrode on the organic light emitting layer; and
forming a metal layer formed on the second electrode layer.

18. The method of claim 16, further comprising:
forming an inorganic composite layer on the metal layer.

19. The method of claim 16,
wherein the metal layer is thicker than 10 nm.

20. The method of claim 16, further comprising:
forming an inorganic layer on the metal layer.

21. The method of claim 16, further comprising:
forming an organic layer and an inorganic layer on the metal layer.

22. The method of claim 20,
wherein the organic layers and the inorganic layers are formed alternatively.

23. The method of claim 16, further comprising:
forming an absorption layer on the metal layer.

24. The method of claim 20,
wherein the organic layer is between about 50 um and about 500 um thick.

25. A display device, comprising:
a substrate;
a transistor;
a pixel electrode that is electrically coupled to the transistor;
a light emitting layer;
a metal layer that has a melting temperature lower than 300° C.; wherein the metal layer protects the light emitting layer from later process.
